(19) United States
(12) Patent Application Publication
(10) Pub. No.: US 2026/0083369 A1
(43) Pub. Date: Mar. 26, 2026

(54) COGNITIVE LOAD ADAPTATION IN MOBILE GENERATIVE ARTIFICIAL INTELLIGENCE

(71) Applicant: International Business Machines Corporation, Armonk, NY (US)

(72) Inventors: Alecio Pedro Delazari Binotto, Munich (DE); Fernando Luiz Koch, Palm Beach Gardens, FL (US); Francis Powlesland, Muenchen (DE); Jose Luis Spagnuolo Sobrinho, Alpharetta, GA (US)

(73) Assignee: International Business Machines Corporation, Armonk, NY (US)

(21) Appl. No.: 18/892,894

(22) Filed: Sep. 23, 2024

(51) Int. Cl. A61B 5/16; A61B 5/00; G06N 3/0475

(52) U.S. Cl. CPC A61B 5/165; A61B 5/7267; A61B 5/7275; G06N 3/0475; A61B 2560/0242

(57) ABSTRACT

An embodiment configures, from an application executing in a mobile device, a sensor to provide a measurement of a factor affecting a user. A value of a cognitive load of the user is inferred from the measurement. An adjustment to a parameter of a content generation model is produced from the application. At the content generation model, a modification in a generated content is caused because of to the adjustment to the parameter. A cognitive load aware content (CLA content) is caused to be output from content generation model because of the modification such that the CLA content causes the cognitive load of the user to remain within a threshold value.

### BACKGROUND

The present invention relates generally to the field of artificial intelligence using Large and Small Language Models, automatic machine learning, and data science. More particularly, the present invention relates to a method, system, and computer program for cognitive load adaptation in mobile generative AI.

Artificial intelligence (AI) technology has evolved significantly over the past few years. Modern AI systems are achieving human-level performance on cognitive tasks like converting speech to text, recognizing objects and images, and translating between different languages. This evolution holds promise for new and improved applications in many industries.

A Large Language Model (LLM or model, plural LLMs or models) is a type of software designed to understand and generate human-like text. LLMs are trained on massive amounts of data from books, articles, websites, and other written sources. At their core, LLMs use a neural network in a transformer architecture that has layers of interconnected nodes that process and interpret text data. An Artificial Neural Network (ANN) is a computing system made up of a number of simple, highly interconnected processing elements, which process information by their dynamic state response to external inputs. ANNs are processing devices (algorithms and/or hardware) that are loosely modeled after the neuronal structure of the mammalian cerebral cortex but on smaller scales. A large ANN implementation of an LLM might have tens of millions of interconnected nodes. By comparison, a mammalian brain has billions of neurons with a corresponding increase in the magnitude of their overall interaction and emergent behavior.

In contrast to an LLM, a small language model (SLM) is a type of machine learning model designed to understand and generate human-like text, but with fewer parameters and less complexity compared to larger language models. Because a SLM is comparatively smaller than an LLM, an SLM requires comparatively less computational power and memory to run, making SLMs more suitable for certain applications and environments, such as on mobile devices.

Small language models might be used in scenarios where resources are limited or where a less sophisticated understanding of language may be sufficient. For example, SLMs might be used for basic chatbots, text completion tasks, or simple automated responses. While they may not have the same depth of understanding or generate as nuanced text as larger models, SLMs can still be quite effective for many practical applications. For example, on a mobile device, and within a limited context of a single user or a small set of users of the device, an SLM may be sufficient to generate content of a limited scope and for the limited consumption of that single user or the small set of users.

Hereinafter, a reference to a model, or to a Language Model (LM), is a reference to either an LLM, or an SLM, or both—LLM and SLM collectively—as may be suitable in a given circumstance. Conventional AI techniques use large data sets to train LMs and use the trained LM to identify patterns and draw conclusions in response to inputs. For example, LMs can analyze the context of the words in a sentence or passage by looking at how words relate to each other in terms of meaning and usage and generate relevant and coherent responses.

AI systems also use LMs as predictive models to perform these functions under different or changing conditions. When given a prompt or question, a model predicts what comes next based on the patterns learned during training. This prediction is generally made word by word, generating responses that aim to be contextually appropriate and informative. After the initial training, LMs can be fine-tuned on specific types of text or for particular tasks to improve their performance in those areas. LMs are designed to mimic human language abilities for tasks like answering questions, writing content, or translating languages.

Classification by an LM refers to the process of categorizing text into predefined classes or categories using a sophisticated neural network-based model with a substantial number of parameters. When provided with a piece of text an LM, which has been trained on a vast amount of text data, processes the input. The LM uses its extensive knowledge of language patterns, context, and semantics to understand the meaning and context of the text. The model extracts features from the text that are relevant for classification. For example, feature extraction might involve identifying keywords, understanding the sentiment, or recognizing specific patterns or contexts. Based on the features extracted, the model assigns the input text to one of the predefined categories. For instance, if the task is sentiment analysis, the categories might be “positive,” “negative,” or “neutral.” For topic classification, the categories could be different subjects or themes relevant to the content. The model performs classification of the input, or portions thereof, and provides the classification result. For example, the model might output a label such as “spam” or “not spam” for an email, or “sports” or “politics” for a news article.

### SUMMARY

The illustrative embodiments provide for training data filtration for large language models. An embodiment includes configuring, from an application executing in a mobile device, a sensor to provide a measurement of a factor affecting a user; inferring, from the measurement a value of a cognitive load of the user; producing, from the application, an adjustment to a parameter of a content generation model; causing, at the content generation model, responsive to the adjustment to the parameter, a modification in a generated content; and causing the content generation model to output, responsive to the modification, a cognitive load aware content (CLA content), wherein the CLA content causes the cognitive load of the user to remain within a threshold value.

Other embodiments of this aspect include corresponding computer systems, apparatus, and computer programs recorded on one or more computer storage devices, each configured to perform the actions of the embodiment.

An embodiment includes a computer-usable program product. The computer-usable program product includes a computer-readable storage medium and program instructions stored on the storage medium.

An embodiment includes a computer system. The computer system includes a processor, a computer-readable memory, and a computer-readable storage medium, and program instructions stored on the storage medium for execution by the processor via the memory.

### BRIEF DESCRIPTION OF THE DRAWINGS

The novel features believed characteristic of the invention are set forth in the appended claims. The invention itself, however, as well as a preferred mode of use, further objectives, and advantages thereof, will best be understood by reference to the following detailed description of the illustrative embodiments when read in conjunction with the accompanying drawings, wherein:

FIG. 1 depicts a block diagram of a computing environment in accordance with an illustrative embodiment;

FIG. 2 depicts a block diagram of an example configuration for cognitive load adaptation in mobile generative AI in accordance with an illustrative embodiment;

FIG. 3 depicts a flowchart of an example overall process for cognitive load adaptation in mobile generative AI in accordance with an illustrative embodiment;

FIG. 4 depicts a flowchart of an example process for multi-modal sensor fusion for contextual information in accordance with an illustrative embodiment;

FIG. 5 depicts a second part of the overall process for cognitive load adaptation in mobile generative AI in accordance with an illustrative embodiment; and

FIG. 6 depicts a part of the overall process for cognitive load adaptation in mobile generative AI in accordance with an illustrative embodiment.

### DETAILED DESCRIPTION

Each individual is unique. The human mind is an incredibly complex tool where different users learn and internalize information differently. Some users need more background to understand information, and some other users can dive right into the details and are bored with background material. The mood of the same user at one time might be more amenable to serious and objective information on a topic, while at another time the user's mood might require a lighter treatment of the same topic.

Different users in the same circumstance might consume the same information differently. For example, different people on a bus might want the route and landmarks information presented to them in different levels of details—depending on their different levels of understanding, different levels of exhaustion, different objectives from the trip, and many other factors. Furthermore, the same person might desire one level of detail at one landmark and a different level of detail at a different landmark on the same trip on the same bus, e.g., due to a personal connection with one landmark and not the other.

The illustrative embodiments recognize that due to these types of differences in a user's demand for content and differences in the manner of consumption of that content under different circumstances, the content generated by AI should also be sensitive to these differences. Furthermore, the illustrative embodiments recognize that the generated content should be sensitive to a content-consumer's personal factors as well as environmental factors. For example, a personal factor may be a level of tiredness or exhaustion, a level of distractedness or attentiveness, a level of health, a level of boredom or interest, a level of intellectual ability or understanding of a topic, etc. And for example, an environmental factor might be a level of ambient noise or temperature, a level of light or darkness, a level of precipitation or humidity, the elevation of a place, exposure to various substances, ability to interact with tactile/audible/visual senses, etc.

Cognitive load refers to the mental effort required by a user to interact with, understand, and effectively use an artificial intelligence system. Cognitive load encompasses how much mental energy is needed to process information, make decisions, and perform tasks while interacting with the AI. If an AI system presents too much information at once or provides data in a complex manner, it can cause information overload and overwhelm a user, increasing cognitive load and making it harder for the user to process and act on the information. The ease with which a user can interact with the AI also affects cognitive load. For example, if an AI requires multiple steps to accomplish a task or involves complex commands, it increases the cognitive load compared to a more streamlined and straightforward interaction. If the AI system frequently makes errors or provides unclear responses, a user may need to spend additional mental effort to correct these issues or find alternative solutions, adding to the user's cognitive load.

The illustrative embodiments recognize that personal factors, environmental factors, or both contribute significantly to a user's cognitive load. The illustrative embodiments recognize that presently, AI-generated content may not consider personal factors, environmental factors, or both from a cognitive load perspective when responding to a demand for content, e.g., in response to a prompt. For situations such as those described above, the illustrative embodiments recognize that AI generated content should be adapted in one or more ways according to one or more personal factors, one or more environmental factors, or some combination thereof that affect a user's cognitive load.

The illustrative embodiments further recognize that such cognitive load-aware adaptation of the generated content is more relevant to mobile users and more needed on mobile devices where the circumstances of content consumption can change significantly from time to time. Presently, mobile device apps are available that implement SLMs to generate all or some content locally on the mobile device by using local as well as external over-the-network sources of underlying information. The illustrative embodiments recognize that providing AI-generated content on mobile devices, which is dynamically matched with the user's real-time cognitive load, is even more difficult due to the comparatively limited computing resources that are generally available on mobile devices. Any reference to “real-time” herein is intended to be as near in time of a measured factor as computationally possible within the prevailing technological constraints.

To help address these and similar problems with generated content, and specifically with cognitive load-aware generated content on mobile devices, the illustrative embodiments provide a system and method for cognitive load adaptation in mobile generative AI. The illustrative embodiments enable dynamic adjustment of generated content based on a user's cognitive load, wherein the information sourcing is adjusted, and the content is generated and adapted in real-time to suit the user's real-time ability to process that information at a given point in time. The illustrative embodiments capture and use both physiological and environmental data to yield a comprehensive context of the user, thus deriving an inference of the user's cognitive load before intelligently making recommendations for content adjustment.

Specifically, the illustrative embodiments address these and similar problems by providing the following three solutions—(i) managing and aggregating sensor data to derive physiological and environmental factors affecting a user's real-time cognitive load and predict future changes therein; (ii) inferring the real-time cognitive load of a user based on physiological and environmental factors in a machine learning model; and (iii) determining appropriate content adjustments based on a combination of the user's real-time cognitive load, historical cognitive load-to-content type mapping, contextual information enhancement in the content, and user feedback for machine learning for improving future content adaptation.

The illustrative embodiments are described with respect to a mobile device, which is a mobile computing platform capable of executing a local generative AI facility (referred to herein as a “local model”), such as an SLM implemented within the mobile computing platform. Furthermore, the local model is configured to communicate over a data network with and utilize another generative AI facility (referred to herein as a “remote model”), such as an LLM operating in a datacenter.

In one embodiment, to determine the user's context—and consequently the user's cognitive load within that context—a local model uses a set of inputs from one or more sensors. A physiological sensor associated with the user provides an input as to the physiological factor that contributes to the user's cognitive load. Some non-limiting examples of physiological sensors, their inputs, and the corresponding cognitive load effects include—a skin conductivity sensor showing a level of perspiration, which is indicative of a level of stress experienced by the user; an eye movement tracker showing pupil dilation and/or rapidity of eye movements, which is indicative of a level of attention or distraction; a heartrate monitor indicating the pulse rate of the user, which is indicative of a level of stress and/or anxiety in the user; a shake/trembling/fidgeting sensor sensing the stability of movement of the user, which is indicative of a level of confusion/fear/hesitation. These examples of the physiological sensors, their possible inputs to an embodiment, and the manner of interpreting those inputs by an embodiment are not intended to be limiting on the illustrative embodiments. From this disclosure, those of ordinary skill in the art will be able to conceive many other sensors, inputs, and interpretations for a similar purpose, and such other conceptions are contemplated within the scope of the illustrative embodiments.

To determine the user's context—and consequently the user's cognitive load within that context, the local model uses a set of inputs from one or more sensors. An environmental sensor associated with the user provides an input as to the environmental factor that contributes to the user's cognitive load. Some non-limiting examples of environmental sensors, their inputs, and the corresponding cognitive load effects include—a camera showing a nearby structure, which is indicative of a type of place being experienced by the user; a microphone capturing ambient sound, which is indicative of noise-induced stress or limited ability to hear audio content; a temperature sensor sensing ambient temperature, which is indicative of a level of irritation/discomfort of the user while in the environment; an accelerometer sensing motion, which is indicative of movement—and therefore the user's ability to perceive content with fine details while in motion. These examples of the environmental sensors, their possible inputs to an embodiment, and the manner of interpreting those inputs by an embodiment are not intended to be limiting on the illustrative embodiments. From this disclosure, those of ordinary skill in the art will be able to conceive many other sensors, inputs, and interpretations for a similar purpose, and such other conceptions are contemplated within the scope of the illustrative embodiments.

In an embodiment, the local model is trained using different types of sensor input data to correspond with the user's cognitive load. The trained local model—when provided real-time inputs from one or more physiological sensors, one or more environmental sensors, or a combination thereof—provides an indication of the user's cognitive load at that time.

In one embodiment, the local model operates alone without the aid of a remote model, and generates cognitive load-adjusted content (CLA content) to be presented on the mobile device in a manner described herein. In another embodiment, the local model operates alone without the aid of a remote model, and the local model and the remote model each generate a portion of CLA content; the local model combines the portions to form complete CLA content that is presented on the mobile device in a manner described herein.

In another embodiment, the local model operates in collaboration with a remote model, the remote model provides either (i) generated non-CLA content, or (ii) information from which to generate content, or (ii) source identification from which to obtain the information to generate content. The local model then uses the provided generated non-CLA content/information and/or source to locally generate CLA content to be presented on the mobile device in a manner described herein.

In another embodiment, the local model operates in collaboration with a remote model and the remote model generates and provides the CLA content to be presented on the mobile device in a manner described herein.

In another embodiment, the local model—operating alone or in collaboration with a remote model—generates a recommendation for cognitive load adjustment. Some other mobile application (e.g., a second local model) accepts the recommendation as one input, generated content as another input, and constructs and provides the CLA content to be presented on the mobile device in a manner described herein.

In one implementation, a local model may execute in a mobile computing environment where the model services multiple users. For example, the mobile computing platform may be a part of a multi-passenger vehicle and different passengers may have different screens on which the local model has to provide user-specific CLA content. In such an implementation, an embodiment described herein can be adapted such that each user and the user's factors are considered distinctly by the local model to provide CLA content for that user on that user's screen. In this manner, an embodiment can be adapted within the scope of the illustrative embodiments to simultaneously provide differently adjusted CLA content to different users from the same local model.

In one embodiment, different local models are available within the mobile computing platform to handle different types of requests from the user. In one case, each local model is capable of generating CLA content in a manner described herein, but in response to different types of requests from the users, in different contexts, or some combination thereof. In another case, each local model is capable of generating raw content but in response to different types of requests from the users, in different contexts, or some combination thereof. Another local model then applies a cognitive load adjustment recommendation to the raw content and generates CLA content in a manner described herein. In such types of multi-model configurations, a local model can be loaded or unloaded (activated or deactivated for operation) within the mobile computing platform depending on the request and/or context.

In one embodiment, a rules-based engine (RBE) processes the sensor inputs to determine the cognitive load of the user. The RBE uses preconfigured rules and/or heuristics to correlate the one or more physiological and environmental sensor inputs with a cognitive load recommendation, which may take the form of a cognitive load metric. The cognitive load metric may specify a type of cognitive load and a value or level within that type. The cognitive load metric can then be used by a content generation model to generate CLA content.

In another embodiment, a local processes the sensor inputs to determine the cognitive load of the user. The local model uses prior training and machine learning to correlate the one or more physiological and environmental sensor inputs with a cognitive load metric. The cognitive load metric may specify a type of cognitive load and a value or level within that type. The cognitive load metric can then be used by a content generation model to generate CLA content. In one embodiment, the present sensor data and the corresponding cognitive load metric output can be used as a machine learning feedback into the local model to improve future cognitive load metric outputs.

A set of one or more cognitive load metric may be output for a given set of sensor inputs. The entire set of the cognitive load metrics or a subset thereof may be used for CLA content generation within the scope of the illustrative embodiments.

In one embodiment, a content generation model (whether a local model, a remote model, or a combination thereof) adjusts the generated content according to the cognitive load recommendation. For example, in one case, the content generation model may determine from the cognitive load recommendation that the user is in a noisy environment, and use this recommendation to deselect some sources of audio information and select some additional sources of corresponding visual information to generate content such that the resulting content is CLA content by virtue of minimizing audio content presentation in the noisy environment and emphasizing visual content which is unaffected by the noise and therefore not cognitive overloading for the user.

In another example case, the content generation model may determine from the cognitive load recommendation that the user is stressed, and use this recommendation to deselect some sources of detailed information and select some additional sources of corresponding summarized information to generate content such that the resulting content is CLA content by virtue of minimizing detailed content presentation under stressful conditions and emphasizing summarized content which minimizes additional cognitive overloading for the user.

In another example case, the content generation model may determine from the cognitive load recommendation that the user is confused or disoriented in the present environment. The CLA content generation model uses this recommendation to select not only the sources of the information that the user requested but also some additional sources of guidance information to generate content such that the resulting content is CLA content by virtue of providing navigation/guidance/guiding information to reorient the user through the user's immediate environment, thereby reducing the user's confusion, which prevents causing cognitive overloading for the user.

In a similar manner, many other cognitive load conditions can be detected and corresponding CLA content generated—e.g., enhancing colored content when light sensitivity is sensed, high contrast content when frustration in low light conditions is detected, larger font while moving, smaller font when stationary, adding background information about the contents of the environment when confused about understanding the environment, changing a navigation path to reduce stressful conditions, prioritizing cheerful information under certain mood conditions, adding entertaining information when boredom is sensed, and many others. From this disclosure, those of ordinary skill in the art will be able to conceive many other cognitive load adjustments and the same are contemplated within the scope of the illustrative embodiments.

For example, one way an embodiment described herein may be implemented is through the “temperature” of a generative AI model or another similarly purposed model setting. For example, in GPT models, temperature is an important but complex setting that affects how the AI generates text. It helps control how creative or random the generated text is, which the illustrative embodiments recognize to be also relevant to the cognitive load. Temperature is one non-limiting example parameter that controls the randomness of the output generated by an AI model and, accordingly, can be used to adjust the cognitive load imposed by the generated content. Temperature and other settings of a model can be used to tune the features in the generated content, such as to prioritize certain types of content, simplify complex information, reduce distracting content, provide additional support features and content, and many others.

For the sake of clarity of the description, and without implying any limitation thereto, the illustrative embodiments are described using some example configurations. From this disclosure, those of ordinary skill in the art will be able to conceive many alterations, adaptations, and modifications of a described configuration for achieving a described purpose, and the same are contemplated within the scope of the illustrative embodiments.

Furthermore, simplified diagrams of the data processing environments are used in the figures and the illustrative embodiments. In an actual computing environment, additional structures or components that are not shown or described herein, or structures or components different from those shown but for a similar function as described herein may be present without departing the scope of the illustrative embodiments.

Furthermore, the illustrative embodiments are described with respect to specific actual or hypothetical components only as examples. Any specific manifestations of these and other similar artifacts are not intended to be limiting to the invention. Any suitable manifestation of these and other similar artifacts can be selected within the scope of the illustrative embodiments.

The examples in this disclosure are used only for the clarity of the description and are not limiting on the illustrative embodiments. Any advantages listed herein are only examples and are not intended to be limiting to the illustrative embodiments. Additional or different advantages may be realized by specific illustrative embodiments. Furthermore, a particular illustrative embodiment may have some, all, or none of the advantages listed above.

Furthermore, the illustrative embodiments may be implemented with respect to any type of data, data source, or access to a data source over a data network. Any type of data storage device may provide the data to an embodiment of the invention, either locally at a data processing system or over a data network within the scope of the invention. Where an embodiment is described using a mobile device, any type of data storage device suitable for use with the mobile device may provide the data to such embodiment, either locally at the mobile device or over a data network, within the scope of the illustrative embodiments.

The illustrative embodiments are described using specific code, computer readable storage media, high-level features, designs, architectures, protocols, layouts, schematics, and tools only as examples and are not limiting to the illustrative embodiments. Furthermore, the illustrative embodiments are described in some instances using particular software, tools, and data processing environments only as an example for the clarity of the description. The illustrative embodiments may be used in conjunction with other comparable or similarly purposed structures, systems, applications, or architectures. For example, other comparable mobile devices, structures, systems, applications, or architectures therefor, may be used in conjunction with such embodiment of the invention within the scope of the invention. An illustrative embodiment may be implemented in hardware, software, or a combination thereof.

The examples in this disclosure are used only for the clarity of the description and are not limiting to the illustrative embodiments. Additional data, operations, actions, tasks, activities, and manipulations will be conceivable from this disclosure and the same are contemplated within the scope of the illustrative embodiments.

Various aspects of the present disclosure are described by narrative text, flowcharts, block diagrams of computer systems, and/or block diagrams of the machine logic included in computer program product (CPP) embodiments. With respect to any flowcharts, depending upon the technology involved, the operations can be performed in a different order than what is shown in a given flowchart. For example, again, depending upon the technology involved, two operations shown in successive flowchart blocks may be performed in reverse order, as a single integrated step, concurrently, or in a manner at least partially overlapping in time.

A computer program product embodiment (“CPP embodiment” or “CPP”) is a term used in the present disclosure to describe any set of one, or more, storage media (also called “mediums”) collectively included in a set of one, or more, storage devices that collectively include machine readable code corresponding to instructions and/or data for performing computer operations specified in a given CPP claim. A “storage device” is any tangible device that can retain and store instructions for use by a computer processor. Without limitation, the computer readable storage medium may be an electronic storage medium, a magnetic storage medium, an optical storage medium, an electromagnetic storage medium, a semiconductor storage medium, a mechanical storage medium, or any suitable combination of the foregoing. Some known types of storage devices that include these mediums include: diskette, hard disk, random access memory (RAM), read-only memory (ROM), erasable programmable read-only memory (EPROM or Flash memory), static random access memory (SRAM), compact disc read-only memory (CD-ROM), digital versatile disk (DVD), memory stick, floppy disk, mechanically encoded device (such as punch cards or pits/lands formed in a major surface of a disc) or any suitable combination of the foregoing. A computer readable storage medium, as that term is used in the present disclosure, is not to be construed as storage in the form of transitory signals per se, such as radio waves or other freely propagating electromagnetic waves, electromagnetic waves propagating through a waveguide, light pulses passing through a fiber optic cable, electrical signals communicated through a wire, and/or other transmission media. As will be understood by those of skill in the art, data is typically moved at some occasional points in time during normal operations of a storage device, such as during access, de-fragmentation or garbage collection, but this does not render the storage device as transitory because the data is not transitory while it is stored.

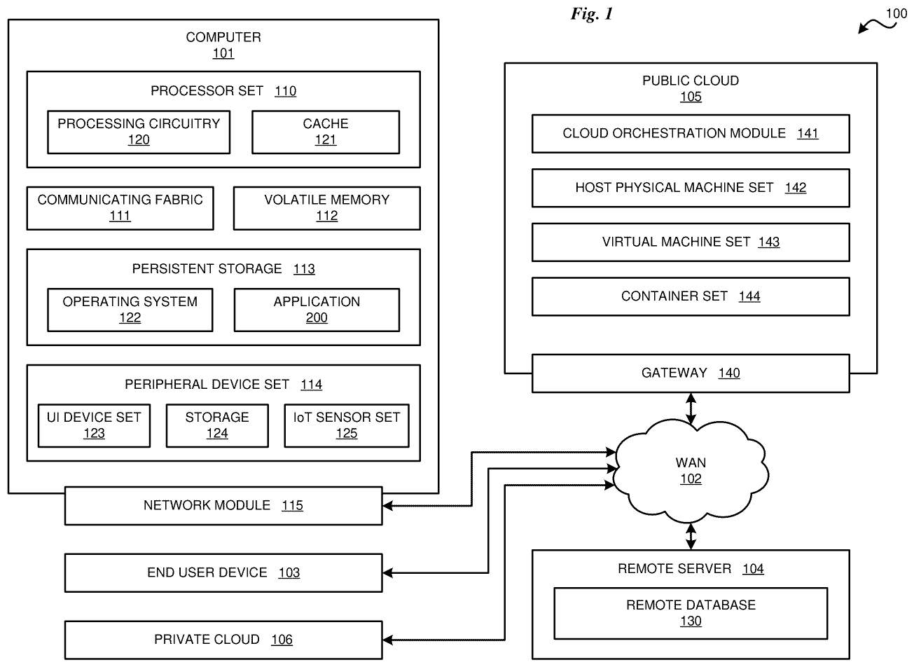

With reference to FIG. 1, this figure depicts a block diagram of a computing environment 100. Computing environment 100 contains an example of an environment for the execution of at least some of the computer code involved in performing the inventive methods, such as application 200 that may execute in mobile computing environment 100 and implement one or more embodiments for cognitive load adaptation in mobile generative AI as described herein. In addition to block 200, computing environment 100 includes, for example, computer 101, wide area network (WAN) 102, end user device (EUD) 103, remote server 104, public cloud 105, and private cloud 106. In this embodiment, computer 101 includes processor set 110 (including processing circuitry 120 and cache 121), communication fabric 111, volatile memory 112, persistent storage 113 (including operating system 122 and block 200, as identified above), peripheral device set 114 (including user interface (UI) device set 123, storage 124, and Internet of Things (IoT) sensor set 125), and network module 115. Remote server 104 includes remote database 130. Public cloud 105 includes gateway 140, cloud orchestration module 141, host physical machine set 142, virtual machine set 143, and container set 144.

COMPUTER 101 may take the form of a desktop computer, laptop computer, tablet computer, smart phone, smart watch or other wearable computer, mainframe computer, quantum computer or any other form of computer or mobile device now known or to be developed in the future that is capable of running a program, accessing a network or querying a database, such as remote database 130. As is well understood in the art of computer technology, and depending upon the technology, performance of a computer-implemented method may be distributed among multiple computers and/or between multiple locations. On the other hand, in this presentation of computing environment 100, detailed discussion is focused on a single computer, specifically computer 101, to keep the presentation as simple as possible. Computer 101 may be located in a cloud, even though it is not shown in a cloud in FIG. 1. On the other hand, computer 101 is not required to be in a cloud except to any extent as may be affirmatively indicated.

PROCESSOR SET 110 includes one, or more, computer processors of any type now known or to be developed in the future. Processing circuitry 120 may be distributed over multiple packages, for example, multiple, coordinated integrated circuit chips. Processing circuitry 120 may implement multiple processor threads and/or multiple processor cores. Cache 121 is memory that is located in the processor chip package(s) and is typically used for data or code that should be available for rapid access by the threads or cores running on processor set 110. Cache memories are typically organized into multiple levels depending upon relative proximity to the processing circuitry. Alternatively, some, or all, of the cache for the processor set may be located “off chip.” In some computing environments, processor set 110 may be designed for working with qubits and performing quantum computing.

Computer readable program instructions are typically loaded onto computer 101 to cause a series of operational steps to be performed by processor set 110 of computer 101 and thereby effect a computer-implemented method, such that the instructions thus executed will instantiate the methods specified in flowcharts and/or narrative descriptions of computer-implemented methods included in this document (collectively referred to as “the inventive methods”). These computer readable program instructions are stored in various types of computer readable storage media, such as cache 121 and the other storage media discussed below. The program instructions, and associated data, are accessed by processor set 110 to control and direct performance of the inventive methods. In computing environment 100, at least some of the instructions for performing the inventive methods may be stored in block 200 in persistent storage 113.

COMMUNICATION FABRIC 111 is the signal conduction path that allows the various components of computer 101 to communicate with each other. Typically, this fabric is made of switches and electrically conductive paths, such as the switches and electrically conductive paths that make up buses, bridges, physical input/output ports and the like. Other types of signal communication paths may be used, such as fiber optic communication paths and/or wireless communication paths.

VOLATILE MEMORY 112 is any type of volatile memory now known or to be developed in the future. Examples include dynamic type random access memory (RAM) or static type RAM. Typically, volatile memory 112 is characterized by random access, but this is not required unless affirmatively indicated. In computer 101, the volatile memory 112 is located in a single package and is internal to computer 101, but, alternatively or additionally, the volatile memory may be distributed over multiple packages and/or located externally with respect to computer 101.

PERSISTENT STORAGE 113 is any form of non-volatile storage for computers that is now known or to be developed in the future. The non-volatility of this storage means that the stored data is maintained regardless of whether power is being supplied to computer 101 and/or directly to persistent storage 113. Persistent storage 113 may be a read only memory (ROM), but typically at least a portion of the persistent storage allows writing of data, deletion of data and re-writing of data. Some familiar forms of persistent storage include magnetic disks and solid state storage devices. Operating system 122 may take several forms, such as various known proprietary operating systems or open source Portable Operating System Interface-type operating systems that employ a kernel. The code included in block 200 typically includes at least some of the computer code involved in performing the inventive methods.

PERIPHERAL DEVICE SET 114 includes the set of peripheral devices of computer 101. Data communication connections between the peripheral devices and the other components of computer 101 may be implemented in various ways, such as Bluetooth connections, Near-Field Communication (NFC) connections, connections made by cables (such as universal serial bus (USB) type cables), insertion-type connections (for example, secure digital (SD) card), connections made through local area communication networks and even connections made through wide area networks such as the internet. In various embodiments, UI device set 123 may include components such as a display screen, speaker, microphone, wearable devices (such as goggles and smart watches), keyboard, mouse, printer, touchpad, game controllers, and haptic devices. Storage 124 is external storage, such as an external hard drive, or insertable storage, such as an SD card. Storage 124 may be persistent and/or volatile. In some embodiments, storage 124 may take the form of a quantum computing storage device for storing data in the form of qubits. In embodiments where computer 101 is required to have a large amount of storage (for example, where computer 101 locally stores and manages a large database) then this storage may be provided by peripheral storage devices designed for storing very large amounts of data, such as a storage area network (SAN) that is shared by multiple, geographically distributed computers. IoT sensor set 125 is made up of sensors that can be used in Internet of Things applications. For example, one sensor may be a thermometer and another sensor may be a motion detector.

NETWORK MODULE 115 is the collection of computer software, hardware, and firmware that allows computer 101 to communicate with other computers through WAN 102. Network module 115 may include hardware, such as modems or Wi-Fi signal transceivers, software for packetizing and/or de-packetizing data for communication network transmission, and/or web browser software for communicating data over the internet. In some embodiments, network control functions and network forwarding functions of network module 115 are performed on the same physical hardware device. In other embodiments (for example, embodiments that utilize software-defined networking (SDN)), the control functions and the forwarding functions of network module 115 are performed on physically separate devices, such that the control functions manage several different network hardware devices. Computer readable program instructions for performing the inventive methods can typically be downloaded to computer 101 from an external computer or external storage device through a network adapter card or network interface included in network module 115.

WAN 102 is any wide area network (for example, the internet) capable of communicating computer data over non-local distances by any technology for communicating computer data, now known or to be developed in the future. In some embodiments, the WAN 012 may be replaced and/or supplemented by local area networks (LANs) designed to communicate data between devices located in a local area, such as a Wi-Fi network. The WAN and/or LANs typically include computer hardware such as copper transmission cables, optical transmission fibers, wireless transmission, routers, firewalls, switches, gateway computers and edge servers.

END USER DEVICE (EUD) 103 is any computer system that is used and controlled by an end user (for example, a customer of an enterprise that operates computer 101), and may take any of the forms discussed above in connection with computer 101. EUD 103 typically receives helpful and useful data from the operations of computer 101. For example, in a hypothetical case where computer 101 is designed to provide a recommendation to an end user, this recommendation would typically be communicated from network module 115 of computer 101 through WAN 102 to EUD 103. In this way, EUD 103 can display, or otherwise present, the recommendation to an end user. In some embodiments, EUD 103 may be a client device, such as thin client, heavy client, mainframe computer, desktop computer and so on.

REMOTE SERVER 104 is any computer system that serves at least some data and/or functionality to computer 101. Remote server 104 may be controlled and used by the same entity that operates computer 101. Remote server 104 represents the machine(s) that collect and store helpful and useful data for use by other computers, such as computer 101. For example, in a hypothetical case where computer 101 is designed and programmed to provide a recommendation based on historical data, then this historical data may be provided to computer 101 from remote database 130 of remote server 104.

PUBLIC CLOUD 105 is any computer system available for use by multiple entities that provides on-demand availability of computer system resources and/or other computer capabilities, especially data storage (cloud storage) and computing power, without direct active management by the user. Cloud computing typically leverages sharing of resources to achieve coherence and economies of scale. The direct and active management of the computing resources of public cloud 105 is performed by the computer hardware and/or software of cloud orchestration module 141. The computing resources provided by public cloud 105 are typically implemented by virtual computing environments that run on various computers making up the computers of host physical machine set 142, which is the universe of physical computers in and/or available to public cloud 105. The virtual computing environments (VCEs) typically take the form of virtual machines from virtual machine set 143 and/or containers from container set 144. It is understood that these VCEs may be stored as images and may be transferred among and between the various physical machine hosts, either as images or after instantiation of the VCE. Cloud orchestration module 141 manages the transfer and storage of images, deploys new instantiations of VCEs and manages active instantiations of VCE deployments. Gateway 140 is the collection of computer software, hardware, and firmware that allows public cloud 105 to communicate through WAN 102.

Some further explanation of virtualized computing environments (VCEs) will now be provided. VCEs can be stored as “images.” A new active instance of the VCE can be instantiated from the image. Two familiar types of VCEs are virtual machines and containers. A container is a VCE that uses operating-system-level virtualization. This refers to an operating system feature in which the kernel allows the existence of multiple isolated user-space instances, called containers. These isolated user-space instances typically behave as real computers from the point of view of programs running in them. A computer program running on an ordinary operating system can utilize all resources of that computer, such as connected devices, files and folders, network shares, CPU power, and quantifiable hardware capabilities. However, programs running inside a container can only use the contents of the container and devices assigned to the container, a feature which is known as containerization.

PRIVATE CLOUD 106 is similar to public cloud 105, except that the computing resources are only available for use by a single enterprise. While private cloud 106 is depicted as being in communication with WAN 102, in other embodiments a private cloud may be disconnected from the internet entirely and only accessible through a local/private network. A hybrid cloud is a composition of multiple clouds of different types (for example, private, community or public cloud types), often respectively implemented by different vendors. Each of the multiple clouds remains a separate and discrete entity, but the larger hybrid cloud architecture is bound together by standardized or proprietary technology that enables orchestration, management, and/or data/application portability between the multiple constituent clouds. In this embodiment, public cloud 105 and private cloud 106 are both part of a larger hybrid cloud.

Measured service: cloud systems automatically control and optimize resource use by leveraging a metering capability at some level of abstraction appropriate to the type of service (e.g., storage, processing, bandwidth, and active user accounts). Resource usage can be monitored, controlled, reported, and invoiced, providing transparency for both the provider and consumer of the utilized service.

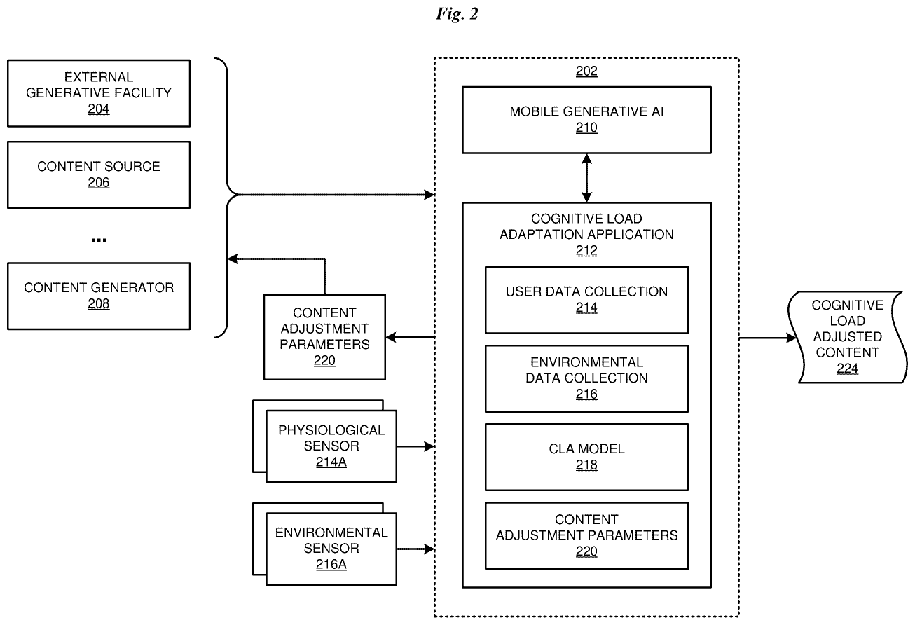

With reference to FIG. 2, this figure depicts a block diagram of an example configuration for cognitive load adaptation in mobile generative AI in accordance with an illustrative embodiment. Application 202 is an example of application 200 in FIG. 1, implemented in computer 101 of FIG. 1, which may be a mobile computing platform. Application 202 is configured to communicate with one or more of external generative facility 204, content source 206, content generator 208, or some combination thereof.

Application 202 comprises mobile generative AI 210, which is an example of a local model as described herein. Mobile generative AI 210 communicates with cognitive load adaptation application 212, which performs a cognitive load adjustment operation in the manner of an embodiment. Module 214 interfaces with and collects a user's physiological sensor data from one or more physiological sensors 214A. Module 216 interfaces with and collects a user's environment data from one or more environmental sensors 216A. cognitive load assessment model 218 uses the data collected by modules 214 and 216 as inputs and produces a cognitive load adjustment recommendation comprising one or more cognitive load adjustment metrics as described herein. Content adjustment parameters 220 are an example of the cognitive load adjustment recommendation.

Mobile generative AI 210 uses content adjustment parameters 220 to produce CLA content 224. In some cases as described herein, one or more of content adjustment parameters 220 may also be exported to an external generative facility 204 and/or content generator 208.

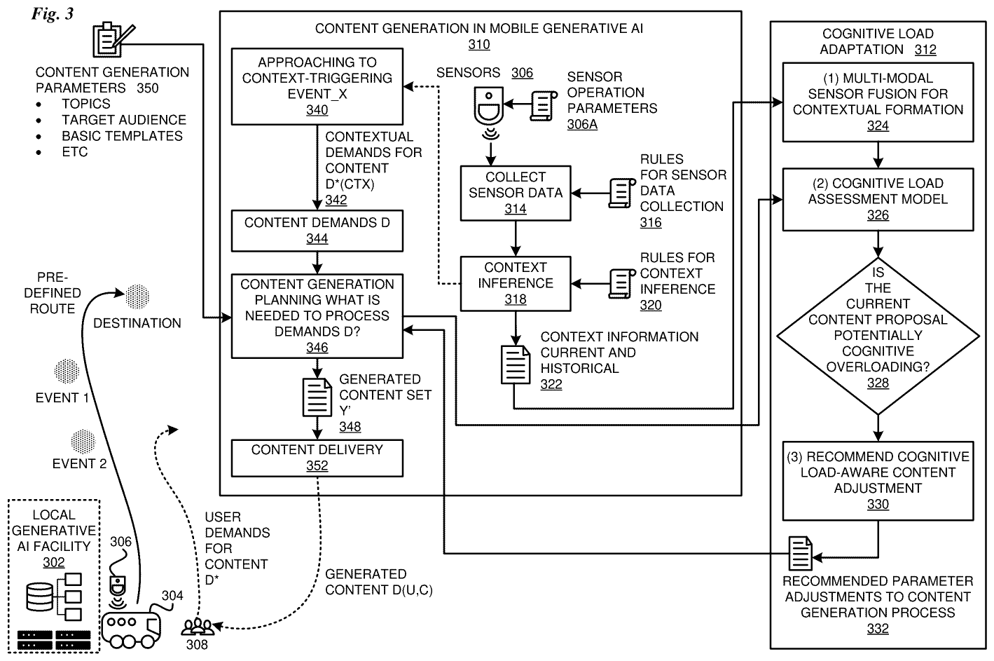

With reference to FIG. 3, this figure depicts a flowchart of an example overall process for cognitive load adaptation in mobile generative AI in accordance with an illustrative embodiment. Local generative facility 302 is an example implementation of application 202 in FIG. 2. Assume that local generative facility 302 is mobile in a multi-passenger vehicle 304, which is configured with one or more sensors 306 relative to users 308 traveling in vehicle 304. Assume that sensors 306 are a combination of physiological and environmental sensors.

Assume that while users 308 are traveling in vehicle 304 along a path where event 1 and event 2 occur on the way to the destination, as shown, user U sends demand D for some content C that has to be generated. Box 310 encapsulates example steps that occur for content generation in the mobile generative AI, e.g., mobile generative AI 210 in FIG. 2. Box 312 encapsulates example steps that occur for cognitive load adaptation, e.g., in cognitive load adaptation application 212 in FIG. 2.

Optionally, in some embodiments, as in this example depiction in FIG. 3, mobile generative AI can be enhanced with some aspects of an embodiment and the remaining aspects implemented in cognitive load adaptation application. Essentially, an aspect or function described herein can be implemented as an enhancement of a local model or in an application that communicates with the local model within the mobile computing platform.

Accordingly, in the depiction of FIG. 3 in box 310, the mobile generative AI is configured to interface with sensors and collect sensor data. Upon demand D by user U, the mobile generative AI communicates with sensor 306 according to sensor operation parameters 306A and collects sensor data (314). Sensor data collection 314 may be performed in conformance with one or more rules (316) for sensor data collection.

The mobile generative AI derives a context inference (318) using the collected sensor data 314 and any rules 320 associated with interpreting sensor data 314 to infer the context. Context inference 322 includes context information that is currently present and optionally, historical context from similar sensor data—if any is available.

The context inference 322 is further processed by cognitive load adaptation application in box 312 with any additional sensor data from multi-modal sensors to refine and form an accurate context 324. The context 324 is used by the cognitive load assessment model 326, e.g., the cognitive load assessment model 218 in FIG. 2. The cognitive load assessment model determines that when the current content proposal (as received from the content generation AI at step 346) is potentially cognitive overloading (328), the cognitive load assessment model should recommend cognitive load-aware content adjustment (330). In such a case, the cognitive load assessment model produces recommended parameter adjustments 332 for the content generation process such that when the parameter adjustments (e.g., the temperature” parameter) are applied to the content generation AI, the resulting content will not cause (or at least mitigate) cognitively overloading for user U.

In box 310, mobile generative AI uses context inference 318 to determine (340) when the mobile computing platform 302 and user U are approaching the context-triggering event, e.g., event 1 along the route. The mobile generative AI computes contextual demands D* (342) (in addition to demand D (344) from the user) that apply to content generation. Using demands D and D*, the mobile generative AI produces content generation planning (346). As noted above, this content generation planning is used by the cognitive load assessment model 326 to apply content adjustments, and any recommendations (332) produced therefrom are fed back to adjust content generation planning (346).

In response to demand D, contextual demand D*, and recommended parameter adjustments 332, the mobile generative AI performs information collection as a result of the adjusted content generation parameters 350. In one example, the content generation parameters may be an adjusted set of topics, target audience settings, changed output templates, or some combination of these and other parameters governing the content output. The mobile generative AI generates content Y′ (352). Content Y′ is the CLA content as described herein, and is delivered to user U.

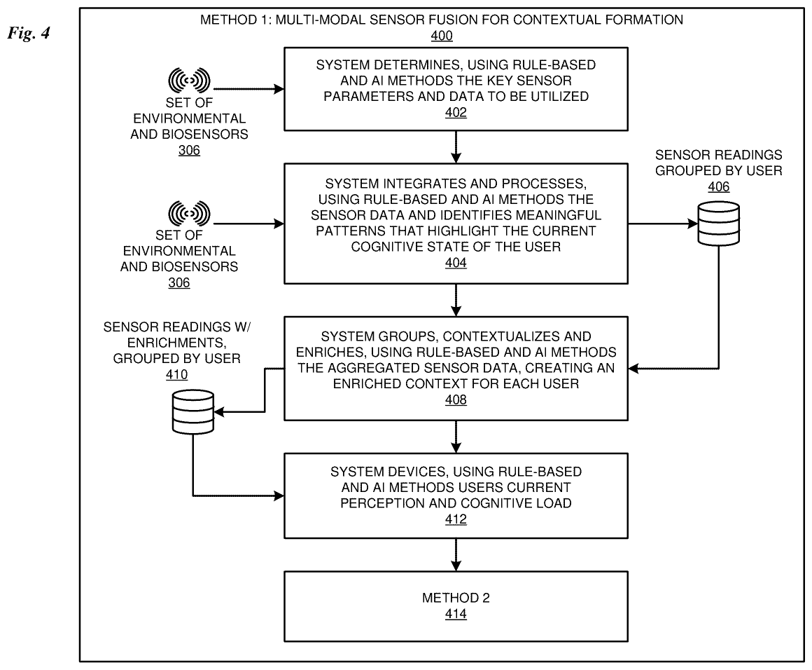

With reference to FIG. 4, this figure depicts a flowchart of an example process for multi-modal sensor fusion for contextual information in accordance with an illustrative embodiment. Process 400 can be implemented in block 324 of FIG. 3 and uses a set of environmental and physiological sensors 306 as described in FIG. 3.

The process determines, using an RBE, an AI model, or some combination thereof, which sensor data are to be used in a given context (402). This determination also drives the selection of sensor parameters 306A corresponding to the selected sensors 306.

The process integrates and processes, again, using an RBE, an AI model, or some combination thereof, the sensor data to determine a pattern that is indicative of the cognitive state of the user (404). Optionally, the process stores in repository (406) the sensor data and the cognitive state determination as historical data for future usage.

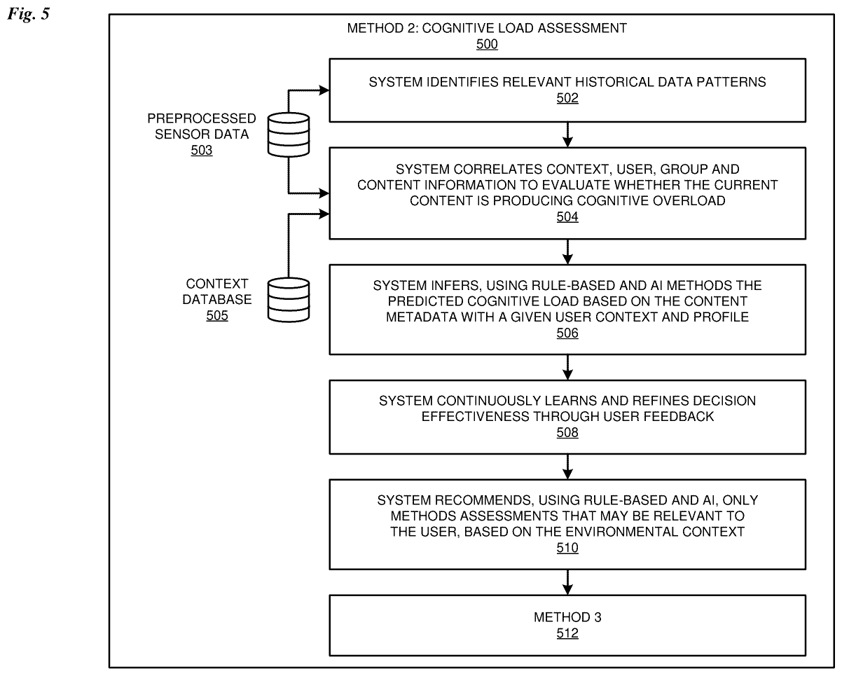

The process uses the determined cognitive state from step 404, and optionally any historically matching cognitive states from repository 406 to determine an enriched context for the user (408). An RBE, an AI model, or some combination thereof may be used to perform step 408. The enriched context as well as the corresponding grouping of sensor data is optionally stored in repository 410 as historical data for future usage. Repositories 406 and 410 may be implemented together or separately. Using the context and sensory data from step 408 and optionally from repository 410 if available, the process derives the user's present cognitive load (412). Process 400 then progresses (414) to the second part of the overall process, which is depicted as process 500 in FIG. 5.

With reference to FIG. 5, this figure depicts a second part of the overall process for cognitive load adaptation in mobile generative AI in accordance with an illustrative embodiment. Process 500 is a continuation from process 400 of FIG. 4 and is the process of cognitive load assessment, as depicted at block 326 in FIG. 3.

Process 500 identifies historical data patterns in the sensor data that is indicative of context, user, user groups, etc. (502). To accomplish this, the process utilizes the current sensor data but may also utilize historical repository 503 of preprocessed sensor data from the past, when such a repository is implemented available. The process correlates the context, the user's sensor data and the content that has been planned to be generated, to determine whether the content will produce a cognitive overload, to wit, a cognitive load exceeding a cognitive load threshold (504). To determine the cognitive load, the process can optionally use historical sensor data and cognitive load correlations that might be available from repository 503, and historical context data and cognitive load correlations that might be available from repository 505.

The process infers, using an RBE, a local model, or some combination thereof, the predicted cognitive load (506). The determination of the cognitive load can be based on the user's determined context and optionally also on the user characteristics if a user profile is available in, or computable from, one of the repositories described herein.

The process optionally continuously learns and refines the effectiveness of the cognitive load-aware content generation (508). This learning is accomplished by capturing user feedback on the accuracy and effectiveness of the CLA content Y′ that is delivered to the user, as depicted in FIG. 3.

Optionally, as depicted in block 510, the process can also limit what factors affect the CLA content. For example, in one case, an illustrative embodiment may be restricted to considering only the environmental factors and not physiological factors in making a CLA content determination.

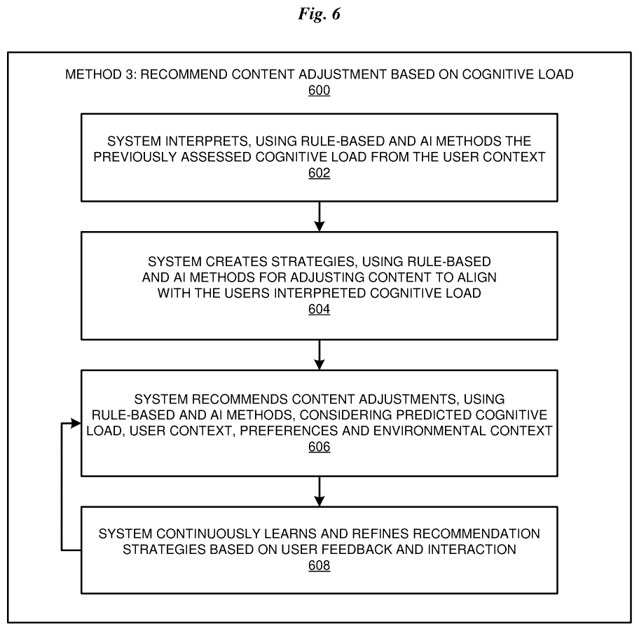

Process 500 then progresses (512) to the third part of the overall process, which is depicted as process 600 in FIG. 6.

With reference to FIG. 6, this figure depicts a part of the overall process for cognitive load adaptation in mobile generative AI in accordance with an illustrative embodiment. Process 600 may be implemented at block 330 in FIG. 3.

Process 600 interprets historical cognitive load of the user from the historical contexts of the user (602). Optionally, the process creates one or more strategies for adjusting generated content to align with the user's interpreted cognitive load (604). The strategies can be stored in a suitable repository for current and future use.

The process recommends content adjustments in consideration of the predicted cognitive load, the user's context, preferences, and the environmental context (606). Optionally, the process can be configured for continuous learning and refinement of the strategies and recommendations through a feedback loop (608).

The following definitions and abbreviations are to be used for the interpretation of the claims and the specification. As used herein, the terms “comprises,” “comprising,” “includes,” “including,” “has,” “having,” “contains,” or “containing,” or any other variation thereof, are intended to cover a non-exclusive inclusion. For example, a composition, a mixture, process, method, article, or apparatus that comprises a list of elements is not necessarily limited to only those elements but can include other elements not expressly listed or inherent to such composition, mixture, process, method, article, or apparatus.

Additionally, the term “illustrative” is used herein to mean “serving as an example, instance or illustration.” Any embodiment or design described herein as “illustrative” is not necessarily to be construed as preferred or advantageous over other embodiments or designs. The terms “at least one” and “one or more” are understood to include any integer number greater than or equal to one, i.e., one, two, three, four, etc. The terms “a plurality” are understood to include any integer number greater than or equal to two, i.e., two, three, four, five, etc. The term “connection” can include an indirect “connection” and a direct “connection.”

References in the specification to “one embodiment,” “an embodiment,” “an example embodiment,” etc., indicate that the embodiment described can include a particular feature, structure, or characteristic, but every embodiment may or may not include the particular feature, structure, or characteristic. Moreover, such phrases are not necessarily referring to the same embodiment. Further, when a particular feature, structure, or characteristic is described in connection with an embodiment, it is submitted that it is within the knowledge of one skilled in the art to affect such feature, structure, or characteristic in connection with other embodiments whether or not explicitly described.

The terms “about,” “substantially,” “approximately,” and variations thereof, are intended to include the degree of error associated with measurement of the particular quantity based upon the equipment available at the time of filing the application. For example, “about” can include a range of ±8% or 5%, or 2% of a given value.

The descriptions of the various embodiments of the present invention have been presented for purposes of illustration but are not intended to be exhaustive or limited to the embodiments disclosed. Many modifications and variations will be apparent to those of ordinary skill in the art without departing from the scope and spirit of the described embodiments. The terminology used herein was chosen to best explain the principles of the embodiments, the practical application or technical improvement over technologies found in the marketplace, or to enable others of ordinary skill in the art to understand the embodiments described herein.

The descriptions of the various embodiments of the present invention have been presented for purposes of illustration but are not intended to be exhaustive or limited to the embodiments disclosed. Many modifications and variations will be apparent to those of ordinary skill in the art without departing from the scope and spirit of the described embodiments. The terminology used herein was chosen to best explain the principles of the embodiments, the practical application or technical improvement over technologies found in the marketplace, or to enable others of ordinary skill in the art to understand the embodiments described herein.

Thus, a computer implemented method, system or apparatus, and computer program product are provided in the illustrative embodiments for managing participation in online communities and other related features, functions, or operations. Where an embodiment or a portion thereof is described with respect to a type of device, the computer implemented method, system or apparatus, the computer program product, or a portion thereof, are adapted or configured for use with a suitable and comparable manifestation of that type of device.

Where an embodiment is described as implemented in an application, the delivery of the application in a Software as a Service (SaaS) model is contemplated within the scope of the illustrative embodiments. In a SaaS model, the capability of the application implementing an embodiment is provided to a user by executing the application in a cloud infrastructure. The user can access the application using a variety of client devices through a thin client interface such as a web browser (e.g., web-based e-mail), or other light-weight client-applications. The user does not manage or control the underlying cloud infrastructure including the network, servers, operating systems, or the storage of the cloud infrastructure. In some cases, the user may not even manage or control the capabilities of the SaaS application. In some other cases, the SaaS implementation of the application may permit a possible exception of limited user-specific application configuration settings.

Embodiments of the present invention may also be delivered as part of a service engagement with a client corporation, nonprofit organization, government entity, internal organizational structure, or the like. Aspects of these embodiments may include configuring a computer system to perform, and deploying software, hardware, and web services that implement, some or all of the methods described herein. Aspects of these embodiments may also include analyzing the client's operations, creating recommendations responsive to the analysis, building systems that implement portions of the recommendations, integrating the systems into existing processes and infrastructure, metering use of the systems, allocating expenses to users of the systems, and billing for use of the systems. Although the above embodiments of present invention each have been described by stating their individual advantages, respectively, present invention is not limited to a particular combination thereof. To the contrary, such embodiments may also be combined in any way and number according to the intended deployment of present invention without losing their beneficial effects.

What is claimed is:

1. A computer-implemented method comprising:
configuring, from an application executing in a mobile device, a sensor to provide a measurement of a factor affecting a user;
inferring, from the measurement a value of a cognitive load of the user;
producing, from the application, an adjustment to a parameter of a content generation model;
causing, at the content generation model, responsive to the adjustment to the parameter, a modification in a generated content; and
causing the content generation model to output, responsive to the modification, a cognitive load aware content (CLA content), wherein the CLA content causes the cognitive load of the user to remain within a threshold value.

2. The computer-implemented method of claim 1, further comprising:
causing, responsive to the adjustment to the parameter, the content generation model to select an alternate source of information; and
using the information from the alternate source in the generated content, wherein the alternate source provides a form of the information, wherein when the form of the information is used in the generated content, the generated content is modified to become the CLA content.

3. The computer-implemented method of claim 1, further comprising:
causing, responsive to the adjustment to the parameter, the content generation model to select an alternate source of information; and
using the information from the alternate source in the generated content, wherein the alternate source provides a level of detail in the information, wherein when the level of detail of the information is used in the generated content, the generated content is modified to become the CLA content.

4. The computer-implemented method of claim 1, further comprising:
correlating, as a part of the inferring, the measurement with the value of the cognitive load using a historical repository of correlations.

5. The computer-implemented method of claim 1, further comprising:
correlating, as a part of the inferring, the measurement with the value of the cognitive load using an AI model executing in the mobile device, wherein the AI model has been trained to predict a cognitive load from an input comprising the measurement.)

6. The computer-implemented method of claim 1, wherein the inferring is performed at the application in the mobile device.

7. The computer-implemented method of claim 1, wherein the inferring is performed at a remote system that is in data communication with the application at the mobile device.

8. The computer-implemented method of claim 1, wherein the content generation model is executing locally in the mobile device.

9. The computer-implemented method of claim 1, wherein the content generation model is executing at a remote system that is in data communication with the application at the mobile device.

10. The computer-implemented method of claim 1, wherein the sensor is a physiological sensor associated with the user, and the physiological sensor provides as the measurement a value of a biometric parameter of the user, wherein the biometric parameter is indicative of the factor.

11. The computer-implemented method of claim 1, wherein the sensor is an environmental sensor associated with the user, and the environmental sensor provides as the measurement a value of a parameter of an environment in which the user is present, wherein the parameter is indicative of the factor.

12. A computer program product comprising:
One or more computer readable storage media; and
program instructions stored on the one or more storage media and configured to perform operations comprising:
configuring, from an application executing in a mobile device, a sensor to provide a measurement of a factor affecting a user;
inferring, from the measurement a value of a cognitive load of the user;
producing, from the application, an adjustment to a parameter of a content generation model;
causing, at the content generation model, responsive to the adjustment to the parameter, a modification in a generated content; and
causing the content generation model to output, responsive to the modification, a cognitive load aware content (CLA content), wherein the CLA content causes the cognitive load of the user to remain within a threshold value.

13. The computer program product of claim 12, further comprising:
causing, responsive to the adjustment to the parameter, the content generation model to select an alternate source of information; and
using the information from the alternate source in the generated content, wherein the alternate source provides a form of the information, wherein when the form of the information is used in the generated content, the generated content is modified to become the CLA content.

14. The computer program product of claim 12, further comprising:
causing, responsive to the adjustment to the parameter, the content generation model to select an alternate source of information; and
using the information from the alternate source in the generated content, wherein the alternate source provides a level of detail in the information, wherein when the level of detail of the information is used in the generated content, the generated content is modified to become the CLA content.

15. The computer program product of claim 12, further comprising:
correlating, as a part of the inferring, the measurement with the value of the cognitive load using a historical repository of correlations.

16. The computer program product of claim 12, further comprising:
correlating, as a part of the inferring, the measurement with the value of the cognitive load using an AI model executing in the mobile device, wherein the AI model has been trained to predict a cognitive load from an input comprising the measurement.)

17. The computer program product of claim 12, wherein the inferring is performed at the application in the mobile device.

18. The computer program product of claim 12, wherein the stored program instructions are stored in a computer readable storage device in a data processing system, and wherein the stored program instructions are transferred over a network from a remote data processing system.

19. The computer program product of claim 12, wherein the stored program instructions are stored in a computer readable storage device in a server data processing system, and wherein the stored program instructions are downloaded in response to a request over a network to a remote data processing system for use in a computer readable storage device associated with the remote data processing system, further comprising:
program instructions to meter use of the program instructions associated with the request; and
program instructions to generate an invoice based on the metered use.

20. A computer system comprising a processor and one or more computer readable storage media, and program instructions collectively stored on the one or more computer readable storage media, the program instructions executable by the processor to cause the processor to perform operations comprising:
configuring, from an application executing in a mobile device, a sensor to provide a measurement of a factor affecting a user;
inferring, from the measurement a value of a cognitive load of the user;
producing, from the application, an adjustment to a parameter of a content generation model;
causing, at the content generation model, responsive to the adjustment to the parameter, a modification in a generated content; and
causing the content generation model to output, responsive to the modification, a cognitive load aware content (CLA content), wherein the CLA content causes the cognitive load of the user to remain within a threshold value.
